(19) United States
(12) Patent Application Publication
(10) Pub. No.: US 2013/0023019 A1
(43) Pub. Date: Jan. 24, 2013

(54) Processing Biomass

(71) Applicant: Marshall Medoff, Brookline, MA (US)

(72) Inventor: Marshall Medoff, Brookline, MA (US)

(73) Assignee: XYLECO, INC., Woburn, MA (US)

(21) Appl. No.: 13/568,962

(22) Filed: Aug. 7, 2012

Related U.S. Application Data
(63) Continuation of application No. PCT/US11/24470, filed on Feb. 11, 2011.
(60) Provisional application No. 61/305,281, filed on Feb. 17, 2010.

(51) Int. Cl. D21B 1/00; D21B 1/36; D21B 1/06; D21H 25/04; C08B 37/00; C12P 7/02; C12P 5/00; C12P 3/00; C12N 9/00; C12N 1/00; C10L 5/00; C12P 7/00

(52) U.S. Cl. 435/132; 44/605; 162/21; 162/50; 162/91; 162/95; 162/97; 162/98; 162/99; 162/100; 162/192; 162/194; 435/155; 435/166; 435/168; 435/183; 435/243; 536/124

(57) ABSTRACT

Biomass (e.g., plant biomass, animal biomass, and municipal waste biomass) is processed to produce useful intermediates and products, such as energy, fuels, foods or materials. For example, systems are described that can use feedstock materials, such as cellulosic and/or lignocellulosic materials, to produce an intermediate or product, e.g., by fermentation.

### RELATED APPLICATIONS

This application is a continuation of International Serial No. PCT/US11/24470 filed Feb. 11, 2011, which claims priority of U.S. Provisional Application Ser. No. 61/305,281 filed Feb. 17, 2010. The entirety of each of these applications is hereby incorporated by reference herein.

### BACKGROUND

Cellulosic and lignocellulosic materials are produced, processed, and used in large quantities in a number of applications. Often such materials are used once, and then discarded as waste, or are simply considered to be waste materials, e.g., sewage, bagasse, sawdust, and stover.

Various cellulosic and lignocellulosic materials, their uses, and applications have been described in U.S. Pat. Nos. 7,074,918, 6,448,307, 6,258,876, 6,207,729, 5,973,035 and 5,952,105; and in various patent applications, including “FIBROUS MATERIALS AND COMPOSITES,” PCT/US2006/010648, filed on Mar. 23, 2006, AND “FIBROUS MATERIALS AND COMPOSITES,” U.S. Patent Application Publication No. 2007/0045456.

### SUMMARY

Generally, this invention relates to carbohydrate-containing materials (e.g., biomass materials or biomass-derived materials), methods of processing such materials to change their structure, and products made from the structurally changed materials. Many of the methods provide materials that can be more readily utilized by a variety of microorganisms to produce useful intermediates and products, e.g., energy, a fuel such as ethanol, a food or a material.

The methods disclosed herein include treating a biomass material to alter the structure of the material by a structural modification treatment other than mechanical treatment, e.g., a treatment selected from the group consisting of radiation, sonication, pyrolysis, oxidation, steam explosion, chemical treatment, and combinations thereof, and then mechanically treating the structurally altered material. In some implementations, one or more of these steps is repeated. For example, the material can be subjected to the structural modification treatment, e.g., irradiated, two or more times, with mechanical treatments between structural modification treatments. In some implementations, the biomass material is initially mechanically treated, e.g., for size reduction, prior to structural modification. The initial and subsequent mechanical treatments may be the same (e.g., shearing followed by further shearing after irradiation), or may be different (e.g., shearing followed by grinding after irradiation).

Without wishing to be bound by any particular theory, it is believed that the structural modification treatment weakens or partially disrupts (e.g., microfractures) the internal crystalline structure of the material, and subsequent mechanical treatment shatters or otherwise further disrupts the weakened structure. This sequence of events reduces the recalcitrance of the feedstock, allowing the treated feedstock to be more readily converted to a product, e.g., a fuel. The optional initial mechanical treatment step can be used to prepare the feedstock material for structural modification, e.g., by reducing the size of the material or “opening up” the material.

It has been found that the total energy requirements to produce a product using the processes described herein are often lower than the total energy requirements of a similar process that includes only structural modification treatment or an initial mechanical treatment followed by structural modification treatment. For example, when one or more mechanical treatments are performed subsequent to structural modification treatment, the structural modification treatment can be performed at a lower energy level with the same or better net effect on recalcitrance. In the case of irradiation, in some implementations a relatively low dose can be delivered to the feedstock, for example less than 60 Mrad, e.g., from about 1 Mrad to about 60 Mrad, or from about 5 Mrad to about 50 Mrad. Thus, the processes described herein may allow an intermediate or a product to be manufactured at relatively low cost using feedstocks that are generally difficult and energy-intensive to process.

However, a wide range of radiation doses can be used. For example, the dose of irradiation can be from about 0.1 Mrad to about 500 Mrad, from about 0.5 Mrad to about 200 Mrad, from about 1 Mrad to about 100 Mrad, or from about 5 Mrad to about 60 Mrad.

In one aspect, the invention features a method that includes mechanically treating a structurally modified biomass feedstock that has been subjected to a structural modification treatment selected from the group consisting of radiation (e.g., electron beam radiation), sonication, pyrolysis, oxidation, steam explosion, chemical treatment, and combinations thereof.

Some implementations may include one or more of the following features. Mechanically treating may include a process selected from the group consisting of cutting, milling, pressing, grinding, shearing and chopping. Milling may include, for example, utilizing a hammer mill, ball mill, colloid mill, conical or cone mill, disk mill, edge mill, Wiley mill or grist mill. In some implementations, structurally modifying includes irradiating, e.g., with an electron beam, alone or in combination with one or more of the other structural modification treatments described herein. Mechanically treating can be performed at ambient temperature, or at a reduced temperature, e.g., as disclosed in U.S. Ser. No. 12/502,629, the complete disclosure of which is incorporated herein by reference. The method may further include repeating the structural modification and mechanical treatment steps one or more times. For instance, the method can include performing an additional structure modifying treatment after mechanically treating.

In some cases, the biomass feedstock comprises a cellulosic or lignocellulosic material. Feedstocks can include, for example, paper, paper products, wood, wood-related materials, particle board, grasses, rice hulls, bagasse, cotton, jute, hemp, flax, bamboo, sisal, abaca, straw, corn cobs, coconut hair, algae, seaweed, microbial materials, altered celluloses, e.g., cellulose acetate, regenerated cellulose, and the like, or mixtures of any of these.

Some methods further include combining the structurally modified, mechanically treated feedstock with a microorganism, the microorganism utilizing the feedstock to produce an intermediate or a product, for example energy, a fuel, e.g., an alcohol, a food or a material. The microorganism can be, for example, a bacterium and/or enzyme. The method can include saccharifying the structurally modified, mechanically treated feedstock, and in some cases fermenting the product of saccharification.

The structurally modified, mechanically treated feedstock has characteristics that can allow it to be readily converted to a product, e.g., by saccharification. For example, in some cases the structurally modified, mechanically treated feedstock has a porosity of at least 80%.

“Structurally modifying” a biomass feedstock, as that phrase is used herein, means changing the molecular structure of the feedstock in any way, including the chemical bonding arrangement, crystalline structure, or conformation of the feedstock. The change may be, for example, a change in the integrity of the crystalline structure, e.g., by microfracturing within the structure, which may not be reflected by diffractive measurements of the crystallinity of the material. Such changes in the structural integrity of the material can be measured indirectly by measuring the yield of a product at different levels of structure-modifying treatment. In addition, or alternatively, the change in the molecular structure can include changing the supramolecular structure of the material, oxidation of the material, changing an average molecular weight, changing an average crystallinity, changing a surface area, changing a degree of polymerization, changing a porosity, changing a degree of branching, grafting on other materials, changing a crystalline domain size, or changing an overall domain size. It is noted that both what is referred to herein as the “structural modification treatment” and the mechanical treatment serve to structurally modify the biomass feedstock. Mechanical treatment does so by the use of mechanical means, while the structural modification means do so using other types of energy (e.g., radiation, ultrasonic energy, or heat) or chemical means.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a block diagram illustrating conversion of biomass into products and co-products.

FIG. 2 is a block diagram illustrating treatment of biomass and the use of the treated biomass in a fermentation process.

### DETAILED DESCRIPTION

Using the methods described herein, biomass (e.g., plant biomass, animal biomass, and municipal waste biomass) can be processed to produce useful intermediates and products such as those described herein. Systems and processes are described below that can use as feedstock materials cellulosic and/or lignocellulosic materials that are readily available, but can be difficult to process by processes such as fermentation. The methods disclosed herein include subjecting a biomass material to a structural modification treatment, e.g., a treatment selected from the group consisting of radiation, sonication, pyrolysis, oxidation, steam explosion, chemical treatment, and combinations thereof, and then mechanically treating the structurally altered material. In some implementations, one or more of these steps is repeated. For example, as will be discussed further below, the material can be irradiated two or more times, with mechanical treatment between irradiation steps. In some implementations, the biomass material is subjected to an initial mechanical treatment prior to the structural modification treatment.

### Systems for Treating Biomass

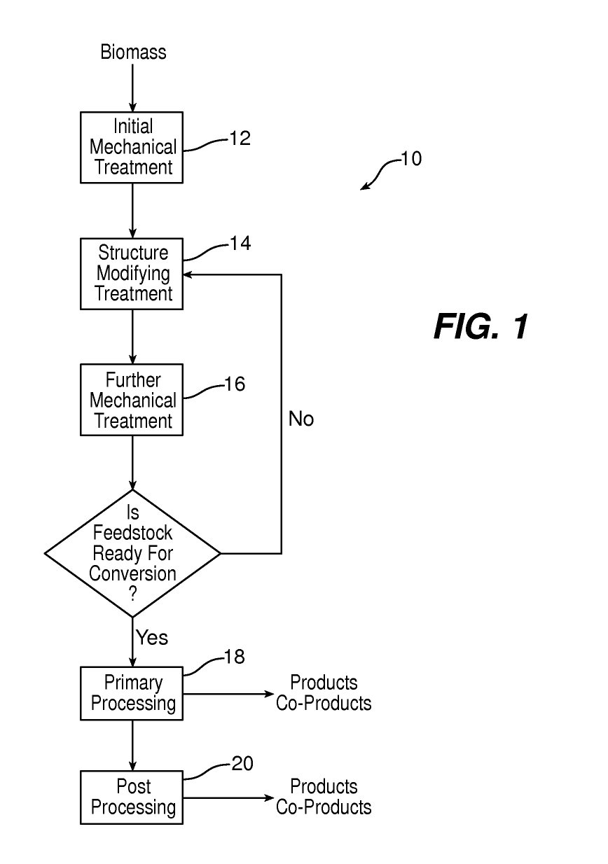

FIG. 1 shows a process 10 for converting biomass, particularly biomass with significant cellulosic and lignocellulosic components, into useful intermediates and products. Process 10 includes initially mechanically treating the feedstock (12), e.g., to reduce the size of the feedstock. The mechanically treated feedstock is then treated with a structure modifying treatment (14) to modify its internal structure, for example by weakening or microfracturing bonds in the crystalline structure of the material. Next, the structurally modified material is subjected to further mechanical treatment (16). This mechanical treatment can be the same as or different from the initial mechanical treatment. For example, the initial treatment can be a size reduction (e.g., cutting) step followed by a shearing step, while the further treatment can be a grinding or milling step.

Without wishing to be bound by any particular theory, it is believed that the structure-modifying treatment disrupts the internal structure of the material, e.g., by micro-fracturing the crystalline structure of the material. The internal structure of the structurally modified material is then further disrupted, e.g., broken, ruptured or fractured, by the subsequent mechanical treatment.

The material can then be subjected to further structure-modifying treatment and mechanical treatment, if further structural change (e.g., reduction in recalcitrance) is desired prior to further processing.

Next, the treated material can be processed with a primary processing step (18), e.g., saccharification and/or fermentation, to produce intermediates and products (e.g., energy, fuel, foods and materials). In some cases, the output of the primary processing step is directly useful but, in other cases, requires further processing provided by a post-processing step (20). For example, in the case of an alcohol, post-processing may involve distillation and, in some cases, denaturation.

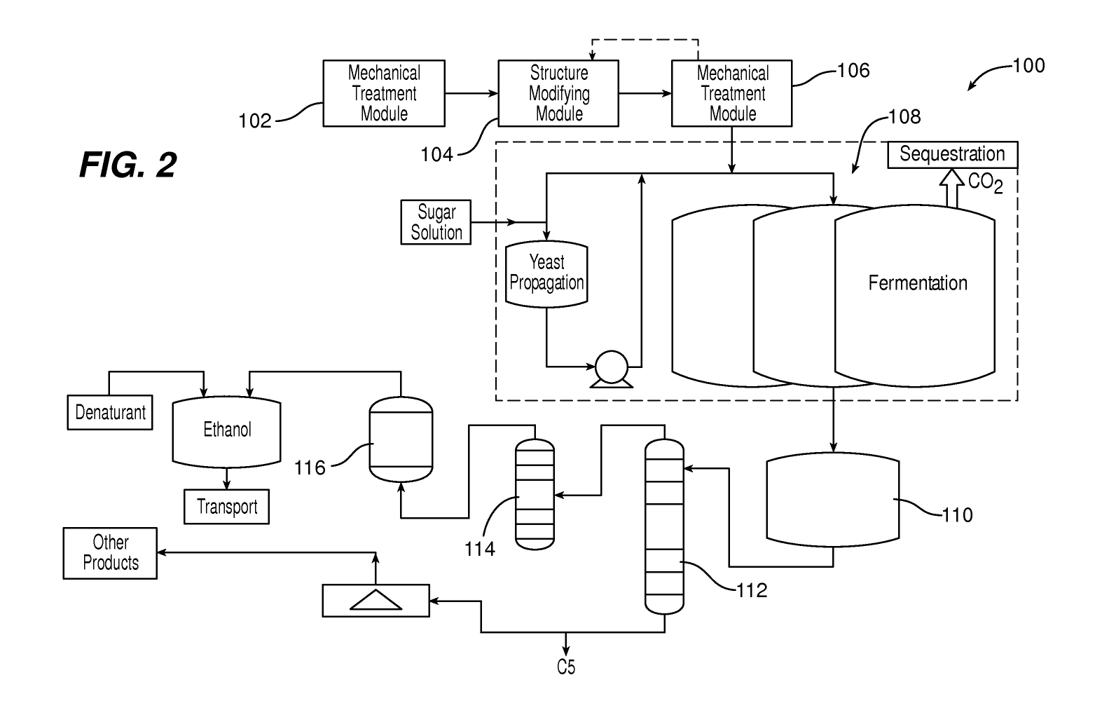

FIG. 2 shows a system 100 that utilizes the steps described above for treating biomass and then using the treated biomass in a fermentation process to produce an alcohol. System 100 includes a module 102 in which a biomass feedstock is initially mechanically treated (step 12, above), a module 104 in which the mechanically treated feedstock is structurally modified (step 14, above), e.g., by irradiation, and a module 106 in which the structurally modified feedstock is subjected to further mechanical treatment (step 16, above). As discussed above, the module 106 may be of the same type as the module 102, or a different type. In some implementations the structurally modified feedstock can be returned to module 102 for further mechanical treatment rather than being further mechanically treated in a separate module 106.

After these treatments, which may be repeated as many times as required to obtain desired feedstock properties, the treated feedstock is delivered to a fermentation system 108. Mixing may be performed during fermentation, in which case the mixing is preferably relatively gentle (low shear) so as to minimize damage to shear sensitive ingredients such as enzymes and other microorganisms. In some embodiments, jet mixing is used, as described in U.S. Ser. No. 61/218,832 and U.S. Ser. No. 61/179,995, the complete disclosures of which are incorporated herein by reference.

Referring again to FIG. 2, fermentation produces a crude ethanol mixture, which flows into a holding tank 110. Water or other solvent, and other non-ethanol components, are stripped from the crude ethanol mixture using a stripping column 112, and the ethanol is then distilled using a distillation unit 114, e.g., a rectifier. Distillation may be by vacuum distillation. Finally, the ethanol can be dried using a molecular sieve 116 and/or denatured, if necessary, and output to a desired shipping method.

In some cases, the systems described herein, or components thereof, may be portable, so that the system can be transported (e.g., by rail, truck, or marine vessel) from one location to another. The method steps described herein can be performed at one or more locations, and in some cases one or more of the steps can be performed in transit. Such mobile processing is described in U.S. Ser. No. 12/374,549 and International Application No. WO 2008/011598, the full disclosures of which are incorporated herein by reference.

Any or all of the method steps described herein can be performed at ambient temperature. If desired, cooling and/or heating may be employed during certain steps. For example, the feedstock may be cooled during mechanical treatment to increase its brittleness. In some embodiments, cooling is employed before, during or after the initial mechanical treatment and/or the subsequent mechanical treatment. Cooling may be performed as described in Ser. No. 12/502,629, the full disclosure of which is incorporated herein by reference. Moreover, the temperature in the fermentation system 108 may be controlled to enhance saccharification and/or fermentation.

The individual steps of the methods described above, as well as the materials used, will now be described in further detail.

### Mechanical Treatments

Mechanical treatments of the feedstock may include, for example, cutting, milling, grinding, pressing, shearing or chopping.

The initial mechanical treatment step may, in some implementations, include reducing the size of the feedstock. In some cases, loose feedstock (e.g., recycled paper or switchgrass) is initially prepared by shearing and/or shredding. In this initial preparation step screens and/or magnets can be used to remove oversized or undesirable objects such as, for example, rocks or nails from the feed stream.

In addition to this size reduction, which can be performed initially and/or later during processing, mechanical treatment can also be advantageous for “opening up,” “stressing,” breaking or shattering the biomass materials, making the cellulose of the materials more susceptible to chain scission and/or disruption of crystalline structure during the structural modification treatment. The open materials can also be more susceptible to oxidation when irradiated.

As discussed above, after irradiation, or other structure-modifying treatment, subsequent mechanical treatment can break bonds within the structure of the material that have been weakened or micro-fractured by the structure-modifying treatment. This further breaking up of the molecular structure of the material tends to reduce the recalcitrance of the material and make it more susceptible to conversion, e.g., by a microorganism such as a bacterium or enzyme.

### Shearing/Screening

In some implementations, the feedstock, either before or after structural modification, is sheared, e.g., with a rotary knife cutter. The feedstock may also be screened. In some embodiments, the shearing of the feedstock and the passing of the material through a screen are performed concurrently.

If desired, the feedstock can be cut prior to the initial mechanical treatment (e.g., shearing), for example using a shredder or other cutter. In some cases, shredding and shearing is accomplished using a combined “shredder-shearer train.” Multiple shredder-shearer trains can be arranged in series, for example two shredder-shearer trains can be arranged in series with output from the first shearer fed as input to the second shredder. Multiple passes through shredder-shearer trains can decrease particle size and increase overall surface area.

### Other Mechanical Treatments

Other methods of mechanically treating the feedstock include, for example, milling or grinding. Milling may be performed using, for example, a hammer mill, ball mill, colloid mill, conical or cone mill, disk mill, edge mill, Wiley mill or grist mill. Grinding may be performed using, for example, a cutting/impact type grinder. Specific examples of grinders include stone grinders, pin grinders, coffee grinders, and burr grinders. Grinding or milling may be provided, for example, by a reciprocating pin or other element, as is the case in a pin mill. Other mechanical treatment methods include mechanical ripping or tearing, other methods that apply pressure to the fibers, and air attrition milling. Suitable mechanical treatments further include any other technique that continues the disruption of the internal structure of the material that was initiated by the previous processing steps.

Suitable cutting/impact type grinders include those commercially available from IKA Works under the tradenames A10 Analysis Grinder and M10 Universal Grinder. Such grinders include metal beaters and blades that rotate at high speed (e.g., greater than 30 m/s or even greater than 50 m/s) within a milling chamber. The milling chamber may be at ambient temperature during operation, or may be cooled, e.g., by water or dry ice.

### Processing Conditions

The feedstock can be mechanically treated in a dry state, a hydrated state (e.g., having up to 10 percent by weight absorbed water), or in a wet state, e.g., having between about 10 percent and about 75 percent by weight water. In some cases, the feedstock can be mechanically treated under a gas (such as a stream or atmosphere of gas other than air), e.g., oxygen or nitrogen, or steam.

It is generally preferred that the feedstock be mechanically treated in a substantially dry condition, e.g., having less than 10 percent by weight absorbed water and preferably less than five percent by weight absorbed water) as dry fibers tend to be more brittle and thus easier to structurally disrupt. In a preferred embodiment, a substantially dry, structurally modified feedstock is ground using a cutting/impact type grinder.

However, in some embodiments the feedstock can be dispersed in a liquid and wet milled. The liquid is preferably the liquid medium in which the treated feedstock will be further processed, e.g., saccharified. It is generally preferred that wet milling be concluded before any shear or heat sensitive ingredients, such as enzymes and nutrients, are added to the liquid medium, since wet milling is generally a relatively high shear process. In some embodiments, the wet milling equipment includes a rotor/stator arrangement. Wet milling machines include the colloidal and cone mills that are commercially available from IKA Works, Wilmington, N.C. (www.ikausa.com).

If desired, lignin can be removed from any feedstock that includes lignin. Also, to aid in the breakdown of the feedstock, in some embodiments the feedstock can be cooled prior to, during, or after irradiation and/or mechanical treatment, as described in Ser. No. 12/502,629, the full disclosure of which is incorporated herein by reference. In addition, or alternatively, the feedstock can be treated with heat, a chemical (e.g., mineral acid, base or a strong oxidizer such as sodium hypochlorite) and/or an enzyme. However, in many embodiments such additional treatments are unnecessary due to the effective reduction in recalcitrance that is provided by the combination of the mechanical and structure modifying treatments.

Characteristics of the Treated Feedstock

Mechanical treatment systems can be configured to produce feed streams with specific characteristics such as, for example, specific bulk densities, maximum sizes, fiber length-to-width ratios, or surface areas ratios.

In some embodiments, a BET surface area of the mechanically treated biomass material is greater than 0.1 m²/g, e.g., greater than 0.25 m²/g, greater than 0.5 m²/g, greater than 1.0 m²/g, greater than 1.5 m²/g, greater than 1.75 m²/g, greater than 5.0 m²/g, greater than 10 m²/g, greater than 25 m²/g, greater than 35 m²/g, greater than 50 m²/g, greater than 60 m²/g, greater than 75 m²/g, greater than 100 m²/g, greater than 150 m²/g, greater than 200 m²/g, or even greater than 250 m²/g.

A porosity of the mechanically treated feedstock, before or after structural modification, can be, e.g., greater than 20 percent, greater than 25 percent, greater than 35 percent, greater than 50 percent, greater than 60 percent, greater than 70 percent, e.g., greater than 80 percent, greater than 85 percent, greater than 90 percent, greater than 92 percent, greater than 94 percent, greater than 95 percent, greater than 97.5 percent, greater than 99 percent, or even greater than 99.5 percent.

The porosity and BET surface area of the material generally increase after each mechanical treatment and after structural modification.

If the biomass material is fibrous, in some implementations, fibers of the mechanically treated material can have a relatively large average length-to-diameter ratio (e.g., greater than 20-to-1), even after the material has been mechanically treated more than once. In addition, the fibers may have a relatively narrow length and/or length-to-diameter ratio distribution.

As used herein, average fiber widths (i.e., diameters) are those determined optically by randomly selecting approximately 5,000 fibers. Average fiber lengths are corrected length-weighted lengths. BET (Brunauer, Emmet and Teller) surface areas are multi-point surface areas, and porosities are those determined by mercury porosimetry.

If the biomass material is fibrous, the average length-to-diameter ratio of fibers of the mechanically treated material can be, e.g., greater than 8/1, e.g., greater than 10/1, greater than 15/1, greater than 20/1, greater than 25/1, or greater than 50/1. An average length of the fibers can be, e.g., between about 0.5 mm and 2.5 mm, e.g., between about 0.75 mm and 1.0 mm, and an average width (i.e., diameter) of the fibers can be, e.g., between about 5 μm and 50 μm, e.g., between about 10 μm and 30 μm.

In some embodiments in which the biomass material is fibrous, a standard deviation of the length of fibers of the mechanically treated material is less than 60 percent of an average length of the fibers, e.g., less than 50 percent of the average length, less than 40 percent of the average length, less than 25 percent of the average length, less than 10 percent of the average length, less than 5 percent of the average length, or even less than 1 percent of the average length.

### Densification

Densified materials can be processed by any of the methods described herein. A mechanically treated feedstock having a low bulk density can be densified to a product having a higher bulk density. For example, a feedstock material having a bulk density of 0.05 g/cm³ can be densified by sealing the material in a relatively gas impermeable structure, e.g., a bag made of polyethylene or a bag made of alternating layers of polyethylene and a nylon, and then evacuating the entrapped gas, e.g., air, from the structure. After evacuation of the air from the structure, the material can have, e.g., a bulk density of greater than 0.3 g/cm³, e.g., 0.5 g/cm³, 0.6 g/cm³, 0.7 g/cm³ or more, e.g., 0.85 g/cm³. After densification, the product can processed by any of the methods described herein. This can be advantageous when it is desirable to transport the material to another location, e.g., a remote manufacturing plant, where the material can be added to a solution, e.g., to saccharify or ferment the material. Any material described herein can be densified, e.g., for transport or storage, and then “opened up” for further processing by any one or more methods described herein. Densification is described, for example, in U.S. Ser. No. 12/429,045, the full disclosure of which is incorporated herein by reference.

### Structural Modification Treatment

The feedstock is subjected to one or more structural modification treatments to modify its structure by, for example, reducing the average molecular weight of the feedstock, changing the crystalline structure of the feedstock (e.g., by microfracturing within the structure which may or may not alter the crystallinity as measured by diffractive methods), and/or increasing the surface area and/or porosity of the feedstock. In some embodiments, structural modification reduces the molecular weight of the feedstock and/or increases the level of oxidation of the feedstock.

Processes that modify the structure of the feedstock include one or more of irradiation, sonication, oxidation, pyrolysis, chemical treatment (e.g., acid or base treatment) and steam explosion. In some preferred implementations, the structure is modified by a process that includes irradiation. When irradiation is used, the process can further include one or more of sonication, oxidation, pyrolysis, chemical treatment, and steam explosion.

### Radiation Treatment

Irradiating the combination can include subjecting the combination to accelerated electrons, such as electrons having an energy of greater than about 2 MeV, 4MeV, 6 MeV, or even greater than about 8 MeV, for example from about 2.0 to 8.0 MeV or from about 4.0 to 6.0 MeV. In some embodiments, electrons are accelerated to, for example, a speed of greater than 75 percent of the speed of light, e.g., greater than 85, 90, 95, or 99 percent of the speed of light.

In some instances, the irradiation is performed at a dosage rate of greater than about 0.25 Mrad per second, e.g., greater than about 0.5, 0.75, 1.0, 1.5, 2.0, or even greater than about 2.5 Mrad per second. In some embodiments, the irradiating is performed at a dose rate of between 5.0 and 1500.0 kilorads/hour, e.g., between 10.0 and 750.0 kilorads/hour or between 50.0 and 350.0 kilorads/hours.

In some embodiments, the irradiating (with any radiation source or a combination of sources) is performed until the material receives a dose of at least 0.1 Mrad, at least 0.25 Mrad, e.g., at least 1.0 Mrad, at least 2.5 Mrad, at least 5.0 Mrad, at least 10.0 Mrad, at least 60 Mrad or at least 100 Mrad. In some embodiments, the irradiating is performed until the material receives a dose of from about 0.1 Mrad to about 500 Mrad, from about 0.5 Mrad to about 200 Mrad, from about 1 Mrad to about 100 Mrad, or from about 5 Mrad to about 60 Mrad. In some embodiments, a relatively low dose of radiation is applied, e.g., less than 60 Mrad.

Radiation can be applied to any sample that is dry or wet, or even dispersed in a liquid, such as water. For example, irradiation can be performed on cellulosic and/or lignocellulosic material in which less than about 25 percent by weight of the cellulosic and/or lignocellulosic material has surfaces wetted with a liquid, such as water. In some embodiments, irradiating is performed on cellulosic and/or lignocellulosic material in which substantially none of the cellulosic and/or lignocellulosic material is wetted with a liquid, such as water.

In some embodiments, any processing described herein occurs after the cellulosic and/or lignocellulosic material remains dry as acquired or has been dried, e.g., using heat and/or reduced pressure. For example, in some embodiments, the cellulosic and/or lignocellulosic material has less than about five percent by weight retained water, measured at 25° C. and at fifty percent relative humidity.

Radiation can be applied while the cellulosic and/or lignocellulosic material is exposed to air, oxygen-enriched air, or even oxygen itself, or blanketed by an inert gas such as nitrogen, argon, or helium. When maximum oxidation is desired, an oxidizing environment is utilized, such as air or oxygen and the distance from the radiation source is optimized to maximize reactive gas formation, e.g., ozone and/or oxides of nitrogen.

Radiation may be applied under a pressure of greater than about 2.5 atmospheres, such as greater than 5, 10, 15, 20, or even greater than about 50 atmospheres.

Irradiating can be performed utilizing an ionizing radiation, such as gamma rays, x-rays, energetic ultraviolet radiation, such as ultraviolet C radiation having a wavelength of from about 100 nm to about 280 nm, a beam of particles, such as a beam of electrons, slow neutrons or alpha particles. In some embodiments, irradiating includes two or more radiation sources, such as gamma rays and a beam of electrons, which can be applied in either order or concurrently.

In some embodiments, energy deposited in a material that releases an electron from its atomic orbital is used to irradiate the materials. The radiation may be provided by 1) heavy charged particles, such as alpha particles or protons, 2) electrons, produced, for example, in beta decay or electron beam accelerators, or 3) electromagnetic radiation, for example, gamma rays, x rays, or ultraviolet rays. In one approach, radiation produced by radioactive substances can be used to irradiate the feedstock. In some embodiments, any combination in any order or concurrently of (1) through (3) may be utilized.

In some instances when chain scission is desirable and/or polymer chain functionalization is desirable, particles heavier than electrons, such as protons, helium nuclei, argon ions, silicon ions, neon ions, carbon ions, phoshorus ions, oxygen ions or nitrogen ions can be utilized. When ring-opening chain scission is desired, positively charged particles can be utilized for their Lewis acid properties for enhanced ring-opening chain scission.

In some embodiments, the irradiated biomass has a number average molecular weight (M_{N2}) that is lower than the number average molecular weight of the biomass prior to irradiation (^{T}M_{N1}) by more than about 10 percent, e.g., 15, 20, 25, 30, 35, 40, 50 percent, 60 percent, or even more than about 75 percent.

In some embodiments, the starting number average molecular weight (prior to irradiation) is from about 200,000 to about 3,200,000, e.g., from about 250,000 to about 1,000,000 or from about 250,000 to about 700,000, and the number average molecular weight after irradiation is from about 50,000 to about 200,000, e.g., from about 60,000 to about 150,000 or from about 70,000 to about 125,000. However, in some embodiments, e.g., after extensive irradiation, it is possible to have a number average molecular weight of less than about 10,000 or even less than about 5,000.

In some instances, the irradiated biomass has cellulose that has as crystallinity (^{T}C₂) that is lower than the crystallinity (^{T}C₁) of the cellulose of the biomass prior to irradiation. For example, (^{T}C₂) can be lower than (^{T}C₁) by more than about 10 percent, e.g., 15, 20, 25, 30, 35, 40, or even more than about 50 percent.

In some embodiments, the starting crystallinity index (prior to irradiation) is from about 40 to about 87.5 percent, e.g., from about 50 to about 75 percent or from about 60 to about 70 percent, and the crystallinity index after irradiation is from about 10 to about 50 percent, e.g., from about 15 to about 45 percent or from about 20 to about 40 percent. However, in some embodiments, e.g., after extensive irradiation, it is possible to have a crystallinity index of lower than 5 percent. In some embodiments, the material after irradiation is substantially amorphous.

In some embodiments, the irradiated biomass can have a level of oxidation (^{T}O₂) that is higher than the level of oxidation (^{T}O₁) of the biomass prior to irradiation. A higher level of oxidation of the material can aid in its dispersability, swellability and/or solubility, further enhancing the materials susceptibility to chemical, enzymatic or biological attack. The irradiated biomass material can also have more hydroxyl groups, aldehyde groups, ketone groups, ester groups or carboxylic acid groups, which can increase its hydrophilicity.

### Ionizing Radiation

Each form of radiation ionizes the biomass via particular interactions, as determined by the energy of the radiation. Heavy charged particles primarily ionize matter via Coulomb scattering; furthermore, these interactions produce energetic electrons that may further ionize matter. Alpha particles are identical to the nucleus of a helium atom and are produced by the alpha decay of various radioactive nuclei, such as isotopes of bismuth, polonium, astatine, radon, francium, radium, several actinides, such as actinium, thorium, uranium, neptunium, curium, californium, americium, and plutonium. When particles are utilized, they can be neutral (uncharged), positively charged or negatively charged. When charged, the charged particles can bear a single positive or negative charge, or multiple charges, e.g., one, two, three or even four or more charges. In instances in which chain scission is desired, positively charged particles may be desirable, in part, due to their acidic nature. When particles are utilized, the particles can have the mass of a resting electron, or greater, e.g., 500, 1000, 1500, or 2000 or more, e.g., 10,000 or even 100,000 times the mass of a resting electron. For example, the particles can have a mass of from about 1 atomic unit to about 150 atomic units, e.g., from about 1 atomic unit to about 50 atomic units, or from about 1 to about 25, e.g., 1, 2, 3, 4, 5, 10, 12 or 15 amu. Accelerators used to accelerate the particles can be electrostatic DC, electrodynamic DC, RF linear, magnetic induction linear, or continuous wave. For example, cyclotron type accelerators are available from IBA, Belgium, such as the Rhodotron® system, while DC type accelerators are available from RDI, now IBA Industrial, such as the Dynamitron®. Exemplary ions and ion accelerators are discussed in Introductory Nuclear Physics, Kenneth S. Krane, John Wiley & Sons, Inc. (1988), Krsto Prelec, FIZIKA B 6 (1997) 4, 177-206, Chu, William T., “Overview of Light-Ion Beam Therapy”, Columbus-Ohio, ICRU-IAEA Meeting, 18-20 March 2006, Iwata, Y. et al., “Alternating-Phase-Focused IH-DTL for Heavy-Ion Medical Accelerators”, Proceedings of EPAC 2006, Edinburgh, Scotland, and Leitner, C. M. et al., “Status of the Superconducting ECR Ion Source Venus”, Proceedings of EPAC 2000, Vienna, Austria.

Electrons interact via Coulomb scattering and bremsstrahlung radiation produced by changes in the velocity of electrons. Electrons may be produced by radioactive nuclei that undergo beta decay, such as isotopes of iodine, cesium, technetium, and iridium. Alternatively, an electron gun can be used as an electron source via thermionic emission.

Electromagnetic radiation interacts via three processes: photoelectric absorption, Compton scattering, and pair production. The dominating interaction is determined by the energy of the incident radiation and the atomic number of the material. The summation of interactions contributing to the absorbed radiation in cellulosic material can be expressed by the mass absorption coefficient (see “Ionization Radiation” in PCT/US2007/022719).

Electromagnetic radiation can be subclassified as gamma rays, x rays, ultraviolet rays, infrared rays, microwaves, or radiowaves, depending on wavelength.

Gamma radiation has the advantage of a significant penetration depth into a variety of material in the sample. Sources of gamma rays include radioactive nuclei, such as isotopes of cobalt, calcium, technicium, chromium, gallium, indium, iodine, iron, krypton, samarium, selenium, sodium, thalium, and xenon.

Sources of x rays include electron beam collision with metal targets, such as tungsten or molybdenum or alloys, or compact light sources, such as those produced commercially by Lyncean.

Sources for ultraviolet radiation include deuterium or cadmium lamps.

Sources for infrared radiation include sapphire, zinc, or selenide window ceramic lamps.

Sources for microwaves include klystrons, Slevin type RF sources, or atom beam sources that employ hydrogen, oxygen, or nitrogen gases.

Electron Beam

In some embodiments, a beam of electrons is used as the radiation source. A beam of electrons has the advantages of high dose rates (e.g., 1, 5, or even 10 Mrad per second), high throughput, less containment, and less confinement equipment. Electrons can also be more efficient at causing chain scission. In addition, electrons having energies of 4-10 MeV can have a penetration depth of 5 to 30 mm or more, such as 40 mm.

Electron beams can be generated, e.g., by electrostatic generators, cascade generators, transformer generators, low energy accelerators with a scanning system, low energy accelerators with a linear cathode, linear accelerators, and pulsed accelerators. Electrons as an ionizing radiation source can be useful, e.g., for relatively thin piles of materials, e.g., less than 0.5 inch, e.g., less than 0.4 inch, 0.3 inch, 0.2 inch, or less than 0.1 inch. In some embodiments, the energy of each electron of the electron beam is from about 0.3 MeV to about 2.0 MeV (million electron volts), e.g., from about 0.5 MeV to about 1.5 MeV, or from about 0.7 MeV to about 1.25 MeV.

In some embodiments, electrons used to treat biomass material can have average energies of 0.05 c or more (e.g., 0.10 c or more, 0.2 c or more, 0.3 c or more, 0.4 c or more, 0.5 c or more, 0.6 c or more, 0.7 c or more, 0.8 c or more, 0.9 c or more, 0.99 c or more, 0.9999 c or more), where c corresponds to the vacuum velocity of light.

Electron beam irradiation devices may be procured commercially from Ion Beam Applications, Louvain-la-Neuve, Belgium or the Titan Corporation, San Diego, CA. Typical electron energies can be 1 MeV, 2 MeV, 4.5 MeV, 7.5 MeV, or 10 MeV. Typical electron beam irradiation device power can be 1 kW, 5 kW, 10 kW, 20 kW, 50 kW, 100 kW, 250 kW, 500 kW, 1000 kW, or even 1500 kW or more. Effectiveness of depolymerization of the feedstock slurry depends on the electron energy used and the dose applied, while exposure time depends on the power and dose. Typical doses may take values of 1 kGy, 5 kGy, 10 kGy, 20 kGy, 50 kGy, 100 kGy, 200 kGy, 500 kGy, 1000 kGy, 1500 kGy, or 2000 kGy.

Tradeoffs in considering electron beam irradiation device power specifications include cost to operate, capital costs, depreciation, and device footprint. Tradeoffs in considering exposure dose levels of electron beam irradiation would be energy costs and environment, safety, and health (ESH) concerns. Tradeoffs in considering electron energies include energy costs; here, a lower electron energy may be advantageous in encouraging depolymerization of certain feedstock slurry (see, for example, Bouchard, et al, Cellulose (2006) 13: 601-610).

It may be advantageous to provide a double-pass of electron beam irradiation in order to provide a more effective depolymerization process. For example, the feedstock transport device could direct the feedstock (in dry or slurry form) underneath and in a reverse direction to its initial transport direction. Double-pass systems can allow thicker feedstock slurries to be processed and can provide a more uniform depolymerization through the thickness of the feedstock slurry.

The electron beam irradiation device can produce either a fixed beam or a scanning beam. A scanning beam may be advantageous with large scan sweep length and high scan speeds, as this would effectively replace a large, fixed beam width. Further, available sweep widths of 0.5 m, 1 m, 2 m or more are available.

Ion Particle Beams

Particles heavier than electrons can be utilized to irradiate carbohydrates or materials that include carbohydrates, e.g., cellulosic materials, lignocellulosic materials, starchy materials, or mixtures of any of these and others described herein. For example, protons, helium nuclei, argon ions, silicon ions, neon ions carbon ions, phoshorus ions, oxygen ions or nitrogen ions can be utilized. In some embodiments, particles heavier than electrons can induce higher amounts of chain scission. In some instances, positively charged particles can induce higher amounts of chain scission than negatively charged particles due to their acidity.

Heavier particle beams can be generated, e.g., using linear accelerators or cyclotrons. In some embodiments, the energy of each particle of the beam is from about 1.0 MeV/atomic unit to about 6,000 MeV/atomic unit, e.g., from about 3 MeV/atomic unit to about 4,800 MeV/atomic unit, or from about 10 MeV/atomic unit to about 1,000 MeV/atomic unit.

Ion beam treatment is discussed in detail in U.S. Ser. No. 12/417,699, the full disclosure of which is incorporated herein by reference.

Electromagnetic Radiation

In embodiments in which the irradiating is performed with electromagnetic radiation, the electromagnetic radiation can have, e.g., energy per photon (in electron volts) of greater than 10² eV, e.g., greater than 10³, 10⁴, 10⁵, 10⁶, or even greater than 10⁷ eV. In some embodiments, the electromagnetic radiation has energy per photon of between 10⁴ and 10⁷, e.g., between 10⁵ and 10⁶ eV. The electromagnetic radiation can have a frequency of, e.g., greater than 10¹⁶ hz, greater than 10¹⁷ hz, 10¹⁸, 10¹⁹, 10²⁰, or even greater than 10²¹ hz. In some embodiments, the electromagnetic radiation has a frequency of between 10¹⁸ and 10²² hz, e.g., between 10¹⁹ to 10²¹ hz.

Combinations of Radiation Treatments

In some embodiments, two or more radiation sources are used, such as two or more ionizing radiations. For example, samples can be treated, in any order, with a beam of electrons, followed by gamma radiation and UV light having wavelengths from about 100 nm to about 280 nm. In some embodiments, samples are treated with three ionizing radiation sources, such as a beam of electrons, gamma radiation, and energetic UV light.

### Quenching and Controlled Functionalization of Biomass

After treatment with one or more ionizing radiations, such as photonic radiation (e.g., X-rays or gamma-rays), e-beam radiation or particles heavier than electrons that are positively or negatively charged (e.g., protons or carbon ions), any of the mixtures of carbohydrate-containing materials and inorganic materials described herein become ionized; that is, they include radicals at levels that are detectable with an electron spin resonance spectrometer. The current practical limit of detection of the radicals is about 10¹⁴ spins at room temperature. After ionization, any biomass material that has been ionized can be quenched to reduce the level of radicals in the ionized biomass, e.g., such that the radicals are no longer detectable with the electron spin resonance spectrometer. For example, the radicals can be quenched by the application of a sufficient pressure to the biomass and/or by utilizing a fluid in contact with the ionized biomass, such as a gas or liquid, that reacts with (quenches) the radicals. The use of a gas or liquid to at least aid in the quenching of the radicals also allows the operator to control functionalization of the ionized biomass with a desired amount and kind of functional groups, such as carboxylic acid groups, enol groups, aldehyde groups, nitro groups, nitrile groups, amino groups, alkyl amino groups, alkyl groups, chloroalkyl groups or chlorofluoroalkyl groups. In some instances, such quenching can improve the stability of some of the ionized biomass materials. For example, quenching can improve the resistance of the biomass to oxidation. Functionalization by quenching can also improve the solubility of any biomass described herein, can improve its thermal stability, which can be important in the manufacture of composites, and can improve material utilization by various microorganisms. For example, the functional groups imparted to the biomass material by quenching can act as receptor sites for attachment by microorganisms, e.g., to enhance cellulose hydrolysis by various microorganisms.

If the ionized biomass remains in the atmosphere, it will be oxidized, such as to an extent that carboxylic acid groups are generated by reaction with the atmospheric oxygen. In some instances with some materials, such oxidation is desired because it can aid in the further breakdown in molecular weight of the carbohydrate-containing biomass, and the oxidation groups, e.g., carboxylic acid groups can be helpful for solubility and microorganism utilization in some instances. However, since the radicals can “live” for some time after irradiation, e.g., longer than 1 day, 5 days, 30 days, 3 months, 6 months or even longer than 1 year, material properties can continue to change over time, which in some instances, can be undesirable.

Detecting radicals in irradiated samples by electron spin resonance spectroscopy and radical lifetimes in such samples is discussed in Bartolotta et al., Physics in Medicine and Biology, 46 (2001), 461-471 and in Bartolotta et al., Radiation Protection Dosimetry, Vol. 84, Nos. 1-4, pp. 293-296 (1999), the contents of each of which are incorporated herein by reference.

### Sonication, Pyrolysis, Oxidation

One or more sonication, pyrolysis, and/or oxidative processing sequences can be used to structurally modify the mechanically treated feedstock. Any of these processes can be used alone or in combination with each other and/or with irradiation. These processes are described in detail in U.S. Ser. No. 12/429,045, the full disclosure of which is incorporated herein by reference.

### Other Processes

Steam explosion can be used alone without any of the processes described herein, or in combination with any of the processes described herein.

Any processing technique described herein can be used at pressure above or below normal, earth-bound atmospheric pressure. For example, any process that utilizes radiation, sonication, oxidation, pyrolysis, steam explosion, or combinations of any of these processes to provide materials that include a carbohydrate can be performed under high pressure, which can increase reaction rates. For example, any process or combination of processes can be performed at a pressure greater than about greater than 25 MPa, e.g., greater than 50 MPa, 75 MPa, 100 MPa, 150 MPa, 200 MPa, 250 MPa, 350 MPa, 500 MPa, 750 MPa, 1,000 MPa, or greater than 1,500 MPa.

### Primary Processes

### Saccharification

In order to convert the treated feedstock to a form that can be readily fermented, in some implementations the cellulose in the feedstock is first hydrolyzed to low molecular weight carbohydrates, such as sugars, by a saccharifying agent, e.g., an enzyme, a process referred to as saccharification. In some implementations, the saccharifying agent comprises an acid, e.g., a mineral acid. When an acid is used, co-products may be generated that are toxic to microorganisms, in which case the process can further include removing such co-products. Removal may be performed using an activated carbon, e.g., activated charcoal, or other suitable techniques.

The materials that include the cellulose are treated with the enzyme, e.g., by combining the material and the enzyme in a solvent, e.g., in an aqueous solution.

Enzymes and biomass-destroying organisms that break down biomass, such as the cellulose and/or the lignin portions of the biomass, contain or manufacture various cellulolytic enzymes (cellulases), ligninases or various small molecule biomass-destroying metabolites. These enzymes may be a complex of enzymes that act synergistically to degrade crystalline cellulose or the lignin portions of biomass. Examples of cellulolytic enzymes include: endoglucanases, cellobiohydrolases, and cellobiases (β-glucosidases). A cellulosic substrate is initially hydrolyzed by endoglucanases at random locations producing oligomeric intermediates. These intermediates are then substrates for exo-splitting glucanases such as cellobiohydrolase to produce cellobiose from the ends of the cellulose polymer. Cellobiose is a water-soluble 1,4-linked dimer of glucose. Finally cellobiase cleaves cellobiose to yield glucose.

### Fermentation

Microorganisms can produce a number of useful intermediates and products by fermenting a low molecular weight sugar produced by saccharifying the treated biomass materials. For example, fermentation or other bioprocesses can produce alcohols, organic acids, hydrocarbons, hydrogen, proteins or mixtures of any of these materials.

Yeast and Zymomonas bacteria, for example, can be used for fermentation or conversion. Other microorganisms are discussed in the Materials section, below. The optimum pH for yeast is from about pH 4 to 5, while the optimum pH for Zymomonas is from about pH 5 to 6. Typical fermentation times are about 24 to 96 hours with temperatures in the range of 26° C. to 40° C., however thermophilic microorganisms prefer higher temperatures.

Mobile fermentors can be utilized, as described in U.S. Provisional Patent Application Serial 60/832,735, now Published International Application No. WO 2008/011598. Similarly, the saccharification equipment can be mobile. Further, saccharification and/or fermentation may be performed in part or entirely during transit.

### Post-Processing

### Distillation

After fermentation, the resulting fluids can be distilled using, for example, a “beer column” to separate ethanol and other alcohols from the majority of water and residual solids. The vapor exiting the beer column can be, e.g., 35% by weight ethanol and can be fed to a rectification column. A mixture of nearly azeotropic (92.5%) ethanol and water from the rectification column can be purified to pure (99.5%) ethanol using vapor-phase molecular sieves. The beer column bottoms can be sent to the first effect of a three-effect evaporator. The rectification column reflux condenser can provide heat for this first effect. After the first effect, solids can be separated using a centrifuge and dried in a rotary dryer. A portion (25%) of the centrifuge effluent can be recycled to fermentation and the rest sent to the second and third evaporator effects. Most of the evaporator condensate can be returned to the process as fairly clean condensate with a small portion split off to waste water treatment to prevent build-up of low-boiling compounds.

### Intermediates and Products

Using, e.g., such primary processes and/or post-processing, the treated biomass can be converted to one or more products, such as energy, fuels, foods and materials. Specific examples of products include, but are not limited to, hydrogen, alcohols (e.g., monohydric alcohols or dihydric alcohols, such as ethanol, n-propanol or n-butanol), sugars, biodiesel, organic acids (e.g., acetic acid and/or lactic acid), hydrocarbons, co-products (e.g., proteins, such as cellulolytic proteins (enzymes) or single cell proteins), and mixtures of any of these. Other examples include carboxylic acids, such as acetic acid or butyric acid, salts of a carboxylic acid, a mixture of carboxylic acids and salts of carboxylic acids and esters of carboxylic acids (e.g., methyl, ethyl and n-propyl esters), ketones, aldehydes, alpha, beta unsaturated acids, such as acrylic acid and olefins, such as ethylene. Other alcohols and alcohol derivatives include propanol, propylene glycol, 1,4-butanediol, 1,3-propanediol, methyl or ethyl esters of any of these alcohols. Other products include methyl acrylate, methylmethacrylate, lactic acid, propionic acid, butyric acid, succinic acid, 3-hydroxypropionic acid, a salt of any of the acids and a mixture of any of the acids and respective salts.

Other intermediates and products, including food and pharmaceutical products, are described in U.S. Provisional Application Ser. No. 12/417,900, the full disclosure of which is hereby incorporated by reference herein.

### Materials

### Biomass Materials

The biomass can be, e.g., a cellulosic or lignocellulosic material. Such materials include paper and paper products (e.g., polycoated paper and Kraft paper), wood, wood-related materials, e.g., particle board, grasses, rice hulls, bagasse, jute, hemp, flax, bamboo, sisal, abaca, straw, corn cobs, coconut hair; and materials high in α-cellulose content, e.g., cotton. Feedstocks can be obtained from virgin scrap textile materials, e.g., remnants, post consumer waste, e.g., rags. When paper products are used they can be virgin materials, e.g., scrap virgin materials, or they can be post-consumer waste. Aside from virgin raw materials, post-consumer, industrial (e.g., offal), and processing waste (e.g., effluent from paper processing) can also be used as fiber sources. Biomass feedstocks can also be obtained or derived from human (e.g., sewage), animal or plant wastes. Additional cellulosic and lignocellulosic materials have been described in U.S. Pat. Nos. 6,448,307, 6,258,876, 6,207,729, 5,973,035 and 5,952,105.

In some embodiments, the biomass material includes a carbohydrate that is or includes a material having one or more β-1,4-linkages and having a number average molecular weight between about 3,000 and 50,000. Such a carbohydrate is or includes cellulose (I), which is derived from (β-glucose 1) through condensation of β(1,4)-glycosidic bonds. This linkage contrasts itself with that for α(1,4)-glycosidic bonds present in starch and other carbohydrates.

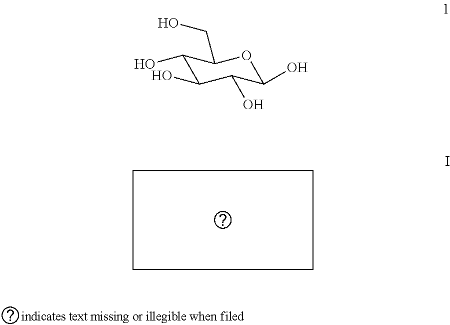 [SMILES: OCC1OC(O)C(O)C(O)C1O]

Starchy materials include starch itself, e.g., corn starch, wheat starch, potato starch or rice starch, a derivative of starch, or a material that includes starch, such as an edible food product or a crop. For example, the starchy material can be arracacha, buckwheat, banana, barley, cassava, kudzu, oca, sago, sorghum, regular household potatoes, sweet potato, taro, yams, or one or more beans, such as favas, lentils or peas. Blends of any two or more starchy materials are also starchy materials.

In some cases the biomass is a microbial material. Microbial sources include, but are not limited to, any naturally occurring or genetically modified microorganism or organism that contains or is capable of providing a source of carbohydrates (e.g., cellulose), for example, protists, e.g., animal protists (e.g., protozoa such as flagellates, amoeboids, ciliates, and sporozoa) and plant protists (e.g., algae such alveolates, chlorarachniophytes, cryptomonads, euglenids, glaucophytes, haptophytes, red algae, stramenopiles, and viridaeplantae). Other examples include seaweed, plankton (e.g., macroplankton, mesoplankton, microplankton, nanoplankton, picoplankton, and femptoplankton), phytoplankton, bacteria (e.g., gram positive bacteria, gram negative bacteria, and extremophiles), yeast and/or mixtures of these. In some instances, microbial biomass can be obtained from natural sources, e.g., the ocean, lakes, bodies of water, e.g., salt water or fresh water, or on land. Alternatively or in addition, microbial biomass can be obtained from culture systems, e.g., large scale dry and wet culture systems.

### Saccharifying Agents

Cellulases are capable of degrading biomass, and may be of fungal or bacterial origin. Suitable enzymes include cellulases from the genera Bacillus, Pseudomonas, Humicola, Fusarium, Thielavia, Acremonium, Chrysosporium and Trichoderma, and include species of Humicola, Coprinus, Thielavia, Fusarium, Myceliophthora, Acremonium, Cephalosporium, Scytalidium, Penicillium or Aspergillus (see, e.g., EP 458162), especially those produced by a strain selected from the species Humicola insolens (reclassified as Scytalidium thermophilum, see, e.g., U.S. Pat. No. 4,435,307), Coprinus cinereus, Fusarium oxysporum, Myceliophthora thermophila, Meripilus giganteus, Thielavia terrestris, Acremonium sp., Acremonium persicinum, Acremonium acremonium, Acremonium brachypenium, Acremonium dichromosporum, Acremonium obclavatum, Acremonium pinkertoniae, Acremonium roseogriseum, Acremonium incoloratum, and Acremonium furatum; preferably from the species Humicola insolens DSM 1800, Fusarium oxysporum DSM 2672, Myceliophthora thermophila CBS 117.65, Cephalosporium sp. RYM-202, Acremonium sp. CBS 478.94, Acremonium sp. CBS 265.95, Acremonium persicinum CBS 169.65, Acremonium acremonium AHU 9519, Cephalosporium sp. CBS 535.71, Acremonium brachypenium CBS 866.73, Acremonium dichromosporum CBS 683.73, Acremonium obclavatum CBS 311.74, Acremonium pinkertoniae CBS 157.70, Acremonium roseogriseum CBS 134.56, Acremonium incoloratum CBS 146.62, and Acremonium furatum CBS 299.70H. Cellulolytic enzymes may also be obtained from Chrysosporium, preferably a strain of Chrysosporium lucknowense. Additionally, Trichoderma (particularly Trichoderma viride, Trichoderma reesei, and Trichoderma koningii), alkalophilic Bacillus (see, for example, U.S. Pat. No. 3,844,890 and EP 458162), and Streptomyces (see, e.g., EP 458162) may be used.

### Fermentation Agents

The microorganism(s) used in fermentation can be natural microorganisms and/or engineered microorganisms. For example, the microorganism can be a bacterium, e.g., a cellulolytic bacterium, a fungus, e.g., a yeast, a plant or a protist, e.g., an algae, a protozoa or a fungus-like protist, e.g., a slime mold. When the organisms are compatible, mixtures of organisms can be utilized.

Suitable fermenting microorganisms have the ability to convert carbohydrates, such as glucose, xylose, arabinose, mannose, galactose, oligosaccharides or polysaccharides into fermentation products. Fermenting microorganisms include strains of the genus Sacchromyces spp. e.g., Sacchromyces cerevisiae (baker's yeast), Saccharomyces distaticus, Saccharomyces uvarum; the genus Kluyveromyces, e.g., species Kluyveromyces marxianus, Kluyveromyces fragilis; the genus Candida, e.g., Candida pseudotropicalis, and Candida brassicae, Pichia stipitis (a relative of Candida shehatae, the genus Clavispora, e.g., species Clavispora lusitaniae and Clavispora opuntiae the genus Pachysolen, e.g., species Pachysolen tannophilus, the genus Bretannomyces, e.g., species Bretannomyces clausenii (Philippidis, G. P., 1996, Cellulose bioconversion technology, in Handbook on Bioethanol: Production and Utilization, Wyman, C. E., ed., Taylor & Francis, Washington, D.C., 179-212).

Commercially available yeasts include, for example, Red Star®/Lesaffre Ethanol Red (available from Red Star/Lesaffre, USA) FALI® (available from Fleischmann's Yeast, a division of Burns Philip Food Inc., USA), SUPERSTART® (available from Alltech, now Lalemand), GERT STRAND® (available from Gert Strand AB, Sweden) and FERMOL® (available from DSM Specialties).

Bacteria may also be used in fermentation, e.g., Zymomonas mobilis and Clostridium thermocellum (Philippidis, 1996, supra).

### OTHER EMBODIMENTS

A number of embodiments of the invention have been described. Nevertheless, it will be understood that various modifications may be made without departing from the spirit and scope of the invention.

For example, the process parameters of any of the processing steps discussed herein can be adjusted based on the lignin content of the feedstock, for example as disclosed in U.S. Provisional Application No. 61/151,724, the full disclosure of which is incorporated herein by reference.

Accordingly, other embodiments are within the scope of the following claims.

What is claimed is:

1. A method comprising:
mechanically treating a structurally modified biomass feedstock that has been subjected to a treatment selected from the group consisting of radiation, sonication, pyrolysis, oxidation, steam explosion, chemical treatment, and combinations thereof.

2. The method of claim 1 wherein mechanically treating comprises a process selected from the group consisting of cutting, milling, grinding, pressing, shearing and chopping.

3. The method of claim 2 wherein mechanically treating comprises grinding.

4. The method of claim 2 wherein mechanically treating comprises milling.

5. The method of claim 4 wherein milling comprises hammer milling.

6. The method of claim 1 wherein the feedstock is subjected to initial mechanical treatment prior to structural modification.

7. The method of claim 6 wherein the initial mechanical treatment is performed at ambient temperature.

8. The method of claim 6 wherein the feedstock is cooled prior to, during, or after the initial mechanical treatment.

9. The method of claim 1 wherein structurally modifying comprises irradiating.

10. The method of claim 9 wherein irradiating comprises irradiating with an electron beam.

11. The method of claim 9 wherein irradiation comprises delivering a dose of from about 0.1 Mrad to about 500 Mrad to the treated material.

12. The method of claim 1 wherein mechanically treating is performed at ambient temperature.

13. The method of claim 1 wherein the feedstock is cooled prior to, during or after mechanically treating.

14. The method of claim 1 wherein mechanically treating is performed above ambient temperature.

15. The method of claim 1 wherein the biomass feedstock comprises a cellulosic or lignocellulosic material.

16. The method of claim 15 wherein the biomass feedstock is selected from the group consisting of paper, paper products, wood, wood-related materials, grasses, switchgrass, rice hulls, bagasse, cotton, jute, hemp, flax, bamboo, sisal, abaca, straw, corn cobs, coconut hair, algae, seaweed, microbial materials, synthetic celluloses, and mixtures thereof.

17. The method of claim 1 further comprising utilizing the structurally modified, mechanically treated feedstock to produce biodiesel.

18. The method of claim 1 further comprising saccharifying the structurally modified, mechanically treated feedstock.

19. The method of claim 18 further comprising fermenting the product of saccharification.

20. The method of claim 19 wherein the product of fermentation comprises hydrogen, an alcohol, an organic acid and/or a hydrocarbon.

21. A composition comprising;
a mechanically treated and structurally modified biomass feedstock, wherein
the feedstock has been structurally modified by a method selected from the group consisting of radiation, sonication, pyrolysis, oxidation, steam explosion, chemical treatment, and combinations thereof.

22. The composition of claim 21 wherein mechanically treating comprises a process selected from the group consisting of cutting, milling, grinding, pressing, shearing and chopping.

23. The composition of claim 21 wherein the composition has a porosity of greater than 80%.

24. The composition of claim 21 further comprising an enzyme or microorganism.
